(19) United States
(12) Patent Application Publication
(10) Pub. No.: US 2010/0028392 A1
(43) Pub. Date: Feb. 4, 2010

(54) Personal Care Article

(76) Inventors: Lee Cawthorne, Hull (GB); Karen Dodd, Hull (GB); Anne Tindal, Hull (GB)

Correspondence Address: TRASKBRITT, P.C./RECKITT BENCKISER, P.O. BOX 2550, SALT LAKE CITY, UT, 84110, US

(21) Appl. No.: 12/226,118

(22) PCT Filed: Apr. 5, 2007
(86) PCT No.: PCT/GB2007/001305

§ 371 Date: Sep. 11, 2009

(30) Foreign Application Priority Data
Apr. 7, 2006 (GB) 0606992.6
Jun. 23, 2006 (GB) 0612486.1

(51) Int. Cl. A61K 8/02; A61K 8/30; A61Q 7/00; A61Q 7/02

(52) U.S. Cl. 424/401; 8/161; 424/70.1

(57) ABSTRACT

A personal care article including a substrate having impregnated therein, applied thereon or arranged to substantially encase, a cosmetic composition which includes a hair modification active, wherein the pH of the composition is substantially less than 9.0, and at least a portion of the article has exfoliating properties.

The present invention is concerned with a personal care article, such as a hair modification article, which is impregnated with a hair modification active. The hair modification active may be a depilatory active and/or a hair regrowth modification active.

The main constituent of hair is keratin a strong hard wearing protein other components are water, pigment, fat, vitamins and trace metals. The outer cuticle layer provides a strong protective layer for the inner cortex. The cortex is made up of a matrix of bundles of protein fibres. Melanin in the cortex provides hair colour. The central hollow core is called the medulla.

Within each fibre there are twists of two α helix protein chains. These protein chains are connecting to each other via sulfur-sulfur (S—S) and hydrogen-hydrogen (H—H) bonds. H—H bonds give hair its flexibility and are broken each time the hair is wetted then reformed when it is dried. S—S bonds form between two keratin chains producing a ladder structure giving the hair its strength (di-sulfide bonds are the strongest bonds in nature).

Articles having a depilatory composition thereon are known to have high pH value; essentially they act as a delivery mechanism for the depilatory composition so that application is easier for the user. Subsequently the user of the article has to rinse away the composition as if it maintains contact with the skin for long periods of time it can be harmful to the skin.

Therefore, according to a first aspect of the present invention, there is provided a personal care article including a substrate having impregnated therein, applied thereon or arranged to substantially encase, a cosmetic composition which includes a hair modification active, wherein the pH of the article is substantially less than 9.0, and at least a portion of the personal care article has abrasive properties.

There is further provided a personal care article including a substrate having impregnated therein, applied thereon or arranged to substantially encase, a cosmetic composition which includes a hair modification active, wherein the pH of the article is substantially less than 9.0, wherein at least a portion of the personal care article has abrasive properties.

As used herein, the term ‘hair modification’ refers to hair removal, hair weakening or hair softening (for example by attacking the di-sulfide bonds in the hair), or by substantially reducing the regrowth of hair.

It is particularly preferred that the article is wet prior to use. The article may be wet through the application of water by the user; alternatively the article may be wet as a result of the cosmetic composition being impregnated on the substrate.

According to a further aspect of the present invention, there is provided use of an article which includes a substrate having impregnated therein, applied thereon or arranged to substantially encase, a composition including a hair modification active, wherein the composition is at a pH substantially less than 9.0, and at least a portion of the personal care article has abrasive properties, in the removal and/or regrowth inhibition of hair.

According to yet a further embodiment of the present invention there is provided a method of modifying mammalian hair which method includes:
a) providing an article according to the first aspect of the present invention; and
b) permitting the article to contact the skin where hair modification is desired.

The method is particularly efficacious if it is carried out regularly by the user, for example, at least once every 2 or 3 days, preferably daily.

Advantageously, the exfoliating properties assist in the modification of hair, for example the removal of hair and/or the retardation of hair regrowth.

A further advantage of the present invention is that the personal care article assists in the reduction of the occurrences of ingrowing hairs on or in the skin. It is believed that the exfoliating properties of the article assist in this reduction of ingrowing hairs.

Accordingly, there is further provided a use of the personal care article substantially as described hereinbefore in the reduction of in growing hairs on mammalian skin.

There is further provided a method of reducing ingrowing hairs on or in mammalian skin which method includes:
a) providing an article according to the first aspect of the present invention; and
b) permitting the article to contact the skin to be treated.

It is particularly preferred that the article is permitted to contact the skin on a regular basis, for example daily or every 2 to 3 days. Advantageously, providing an article that has exfoliation properties enhances the performance of the active present in the composition. The exfoliation properties assist in the removal of hair which has typically been weakened by the active through weakening of the di-sulfide bonds.

It is envisaged that the exfoliation properties may result from the physical properties of the substrate. For example, the substrate may have apertures therein, indentations therein or protrusions thereon. It is, of course, envisaged that the substrate may be manufactured from two or more layers and that the physical properties of the substrate providing exfoliation are present on at least one layer (typically an outermost layer).

It is also envisaged that the substrate may be in the form of a mesh (such as a mesh sheet or a mesh sponge) or a synthetic or natural sponge.

Alternatively, the exfoliation properties may result from at least one exfoliating component in the composition which is impregnated on the substrate, applied on a surface of the substrate, or arranged to encase the composition. It is envisaged that the composition may include two or more exfoliating components.

The exfoliating component may include one or more of a liquid wax (such as jojoba oil or beads), alpha hydroxy acids or beta hydroxy acids (such as salicylic acid), or Silica agglomerates.

It is particularly preferred that the exfoliating component breaks down on the skin.

Preferably, the pH of the composition is less than 8.5, further preferably less than 8.25, such as less than 8.0. Preferably, the pH is greater than 5.0, further preferably greater than 5.5, such as greater than 6.0. It is particularly preferred that the pH is greater than 6.5

It is particularly preferred that the pH is in the range 7 to 8. It is particularly preferred that the composition has a pH which may be tolerated by a user's skin. Advantageously, the use of a substantially neutral pH permits the user to allow the composition to remain on the skin indefinitely without the necessity to rinse the composition from the skin. This is particularly advantageous to the user as it results in a more convenient product to use.

It is believed that many hair modification actives may have both depilatory and hair re-growth inhibition properties. It is further believed that the amount of hair modification active present in a composition and/or the pH of the hair modification composition determines whether the composition acts as a hair re-growth inhibition composition or a depilatory composition.

Accordingly, the hair modification composition may act as a depilatory composition or a hair re-growth inhibition composition, alternatively it may soften or weaken the hair.

The hair modification active may therefore be selected from, but not restricted to, one or more of a sulfur compound, such as potassium thioglycolate, sodium thioglycolate, ammonium thioglycolate, dithioetythritol, thioglycerol, thioglycol, thioxanthine, thiosalicylic acid, N-acetyl-L-cysteine, lipic acid, NaHSO3, Li2S, Na2S, K2S, MgS, CaS, SrS, BaS, (NH4)2S, sodium dihydrolipoate 6, 8-dithiooctanoate, sodium 6,8-dithiooctanoate, salts of hydrogen sulfide (e.g. NaSH or KSH), thioglycolic acid, thioglycerol, 2-mercaptopropionic acid, 3-mercaptopropionic acid, mercaptoethanol, dithioerythritol (DTE), gluatothione (reduced form), thiomalic acid, calcium thioglycolate, guanidine thioglycolate, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homo-cysteine, cysteine, acetyl cysteine, glutathione, dithiothreitol, dihydrolipoic acid, 1,3-dithiopropanol, thioglycolamide, glycerolmonothioglycolate, thioglycolhydrazine, keratinase, hydrazine sulfate, hydrazine disulfate triisocyanate, guanidine thioglycolate, thiosalysilic acid, calcium thioglycolate, cysteamine, xyleine, lipoic acid, sodium dihydrolipoate, thiolactic acid, ammonium thiolactate, monoethanolamine thiolactate, thiopropionic acid, 2 thiolhistidene, 6 mercaptopurine, dimercaptosuccinic acid, thiophenol, 4-methoxythiophenol, 4 bromothiophenol, benzyl mercaptan, 2 mercaptobenzothiazole, bromohexine, carbocysteine, domiodol, erdosteine, letosteine, lysozyme, mecysteine hydrochloride, mesna, sobrerol, stepronin, tiopronin, tyloxapol, nor-dihydroguaiaretic acid (NDGA), thioglycolhydrazide, resveratrol, wortmannin, glutathione (reduced form), dimercaptosuccinic acid and/or Bowman Birk inhibitor (or a mutated form of the BBI). It is, of course, envisaged that blends of more than one hair modification active may be used.

Each hair modification active present in the composition is typically present in an amount less than 10% by weight of the composition, typically less than 7.5% by weight of the composition. It is particularly preferred that the hair modification active is present in an amount less than 5% by weight of the composition. When more than one hair modification active is included in the composition, it is envisaged that the total amount of hair modification active does not exceed 15% by weight of the composition. It is, however, envisaged that the amount of hair modification active present in the composition varies with each specific active used.

The substrate is preferably manufactured from an absorbent or a porous material. The use of an absorbent or porous material advantageously assists in maintaining the composition in or on the substrate.

The substrate may include, but is not limited to, non-woven substrates, woven substrates, thermo-bonded substrates, air laid substrates. The substrate preferably includes a composition which includes Viscose, polyester, polypropylene, polyactic acid polymers, PP Hy, Cotton, “pulp” or vynilin.

It is, however, also envisaged that the substrate may include natural sponges, synthetic sponges, polymeric netted meshes, and the like. Typically, when the substrate is a polymeric netted mesh, it is preferably in the form of a mesh sponge. The mesh sponge may be arranged to substantially encase the composition which is preferably in solid form.

The substrate is preferably flushable and/or biodegradable.

The term ‘non-woven’ is meant that the substrate, or at least a layer of the substrate, is comprised of fibres which are not woven into a fabric and are therefore formed into a sheet, mat, or pad layer. The fibres can either be random (i.e., randomly aligned) or they can be carded (i.e. combed to be oriented in primarily one direction). Furthermore, the non-woven substrate can include a combination of layers of random and carded fibres.

Non-woven substrates may include a variety of natural and/or synthetic materials. By “natural” is meant that the materials are derived from plants, animals, insects or byproducts of plants, animals, and insects. By “synthetic” is meant that the materials are obtained primarily from various man-made materials or from natural materials which have been further altered. Non limiting examples of natural materials useful in the present invention are silk fibres, keratin fibres (such as wool fibres, camel hair fibres) and cellulosic fibres (such as wood pulp fibres, cotton fibres, hemp fibres, jute fibres, flax fibres, and mixtures thereof).

Examples of synthetic materials include, but are not limited to, those selected from the group containing of acetate fibres, acrylic fibres, cellulose ester fibres, modacrylic fibres, polyamide fibres, polyester fibres, polyolefin fibres, polyvinyl alcohol fibres, rayon fibres, polyurethane foam, and mixtures thereof.

It is also envisaged that the substrate includes more than one layer; each layer may be manufactured from a different material. For example, one layer could be of a synthetic material whereas a second layer may be of a natural material.

In one embodiment, the substrate is preferably paper based. Such substrates typically include cellulose-based fibres or filaments from plant cellular sources (pulp).

The substrate preferably has a high wet strength or firmness. The high strength or firmness may result from the addition of binding materials, such as wet strength resins, or the material may be made of staple fibres, e.g. based on cotton, wool, linen and the like.

When the substrate is a woven substrate, it is envisaged that they may include woven cotton and polyester substrates. Suitable woven substrates include towels and articles of clothing.

It is particularly preferred that the substrate is in the shape of a substantially flat sheet such as a wipe, a towlette, a towel, or the like. It is envisaged that the shape of the substrate may be square, circular, oval, or rectangular.

It is envisaged that one or more surfactants may be included in the composition. When a surfactant is present it typically results in the composition being foamable, either by repeated touch or by addition of water.

Preferably, the surfactants are non-ionic surfactants which have structures in which the hydrophilic region contains many oxygen atoms, as hydroxyl or ether groups, which can hydrogen bond to water. They are mostly used as emulsifiers and solubilisers in cosmetic applications (e.g. ceteareth-20, PPG-15 stearyl ether). They may also be amphoteric surfactants which can carry both a positive and negative charge. They are typically used to enrich the texture of formulations and to improve the mildness on personal care products (e.g. cocoamidopropyl betaine). Furthermore, there may be cationic surfactants which have quaternary ammonium ions that are sufficiently polar to attract water and as such provide moisturising capabilities (e.g. polyquaternium 7.)

When a surfactant is present, it is typically present in an amount less than 1.5% by weight of the composition, typically less than 1% by weight of the composition, such as less than 0.5% by weight of the composition. It is particularly preferred that the surfactant is present in an amount less than 0.25% by weight of the composition. Preferably the surfactant is present in an amount greater than 0.025% by weight of the composition, typically more than 0.05% by weight of the composition, such as 0.075% by weight of the composition. It is particularly preferred that the surfactant is present in an amount greater than 0.1% by weight of the composition.

Optionally, the composition includes at least one accelerator that will accelerate the keratin degradation reaction such as urea, thiourea, dimethyl, isosorbide (DMI), ethoxydiglycol (Transcutol) and/or methyl propyl diol (MP diol). The accelerator is typically present in an amount less than 10% by weight of the composition, preferably less than 9% by weight of the composition. Typically the accelerator is present in an amount more than 5% by weight of the composition, preferably more than 6% by weight of the composition. It is particularly preferred that the accelerator is present in the range 7 to 9.0% by weight of the composition, more preferably 7.5 to 8.5% by weight of the composition.

The composition may also include a fragrance. The fragrance may be present in an amount less than 5% by weight of the composition, preferably 0.1 to 2% by weight of the composition, for example from 0.5 to 1.5% by weight of the composition.

The composition may optionally include a dye. The dye may be present in an amount of 0.001% to 5% by weight of the composition, for example 0.001% to 1% by weight % of the composition.

The composition may optionally include a skin conditioning active. Preferably the conditioning active is an emollient which includes one or more of naturally derived oils and esters thereof (e.g. almond oil, hydrogenated castor oil), silicones (e.g. dimethicone PEG-7 Avocadoate), benzoates (e.g. alkyl benzoates), alkanes (e.g. mineral oil), alkenes, alkyl alkanoates (e.g. diisocetyl dodecanedioate), cetearyl esters (e.g. cetearyl isononanoate) acid glycol esters, alkylene glycols and poly-esters and poly-ethers thereof (e.g. polyethylene glycol ether, polypropylene glycol ethers, PEG esters, PPG esters), glycerins, glycols, glycerides, glyceryl triesters (e.g. Triisostearin), alcohols (e.g. C14-15 alcohols, myristyl alcohol, cetearyl alcohol), alkoxylated alcohols (e.g. ceteareth-20, palmeth-2), lactates (e.g. isostearyl lactate), polysorbates (e.g. polysorbate 20), palmitates (e.g. methyl palmitate 1,3 diol, isopropyl palmitate, cetyl palmitate), succinates (e.g. isododecenylsuccinate), laurates (e.g. glyceryl laurate, polyglyceryl-4 laurate), oleates (e.g. ethyl oleates, glyceryl oleates), tocopherol and esters thereof, alkyl acetates, linoelates (e.g. glyceryl linoleates), linoenate (e.g. glyceryl linoenate), myristates (e.g. isostearyl myristate), sugars (e.g. xylitol, glucose), citrates (e.g. dilauryl citrate), carbonates (e.g. diethylhexyl carbonate), phosphates (e.g. palmeth-2 phosphate), dehydro-acetic acid, stearates (e.g. glyceryl stearate). Skin conditioning actives or emollients may be present in an amount of from 0.01% to 10% by weight of the composition. Preferably, the skin conditioning active is present in an amount of 0.01% to 5% by weight of the composition, for example, 0.1% to 1% by weight of the composition.

Advantageously, the inclusion of a skin condition active provides a further (secondary) benefit of the article by moisturising and/or conditioning the skin. The addition of the skin conditioning agents advantageously provides such secondary benefits as unlike conventional depilatory formulations, the composition is typically not washed off or removed from the skin during/after use. Therefore, the skin conditioning agent is permitted to remain in contact with the skin so as to provide the beneficial skin conditioning properties.

Accordingly, the article according to the present invention has the combined effect of modifying hair growth and moisturising the skin.

The composition may also include a preservative. The preservative may be present in an amount of 0.01 to 5% by weight, for example, 0.1 to 1% by weight. Suitable preservatives include Parabens and sodium salts thereof, isothiazolinones, phenolics (e.g. phenoxyethanol), alcohols, halogenated compounds (e.g. methyldibromo glutaronitrile, iodopropynyl butylcarbamate), quaternary compounds (e.g. benzalkonium chloride, polyaminopropyl biguanide), natural compounds (e.g. formaldehyde, tea tree oil), formaldehyde donors (e.g. diazolidinyl urea, DMDM hydantoin), chelating agents (e.g. EDTA), glycols (e.g. caprylyl glycol, butylene glycol), acids and salts and esters thereof (e.g. benzoic acid, sorbic acid), anti oxidants (e.g. BHA, tocopherol).

It is particularly preferred that the composition is an aqueous based composition. However, when the substrate (such as a sponge, a cloth or a mesh sponge) is arranged to encase the composition, the composition is preferably substantially solid.

According to a further aspect of the present invention, there is provided a hair modification composition having a pH of substantially less than 9.0. The hair modification composition is substantially as described hereinbefore.

Accordingly to yet a further aspect of the present invention there is provided a hair modification composition, substantially as described hereinbefore, for use substantially simultaneously with a substrate.

The substrate is typically substantially as described hereinbefore.

According to still yet a further embodiment of the present invention there is provided a mammalian hair modification kit comprising:
a) a hair modification composition having a pH of substantially less than 9.0; and
b) an applicator for applying to, or blending on, the composition the skin.

### EXAMPLE 1

The present invention will now be described by way of example only.

An aqueous solution containing 5% w/w of depilatory active was impregnated on to a viscose/rayon substrate with inherent exfoliating properties. An in-vitro hair removal efficacy test was carried out by securing hairs in a vice and applying the active by rubbing the impregnated wipe over the hairs that were protruding at the vice's surface. Use of the wipe impregnated with the composition containing active was carried out daily.

Results

| Day | % Hair removed |
|---|---|
| 1 | 59% |
| 2 | 89% |
| 3 | 93% |
| 4 | 100% |

A direct comparison was carried out using a wipe impregnated with a composition that did not contain an active following 4 days of treatment only 3.4% of hairs were removed.

### EXAMPLE 2

A formulation having the following composition was impregnanted onto a substrate having a rough or abrasive surface.

Formulation:

| | |
|---|---|
| DI water | 78.23 |
| NAC | 2.85 |
| Emulpharma CM3 | 10.15 |
| Lotus Flower Milk | 0.1 |
| Cremophor CO410 | 4.05 |
| Glycerin | 2.095 |
| Phenonip XB | 1.37 |
| Fragrance | 0.3 |

An in-vivo efficacy test was carried out by 80 people. Firstly, the candidates removed all hair from their legs by shaving. Each candidate then used the wipe impregnated with the composition daily. Comments of the test candistes after a 2 week period were as follows:

| | |
|---|---|
| Smoother than normal | 100% |
| Skin Softer than normal | 96.25% |
| Less hair stubble than normal | 83.75% |
| Stops stubbly feeling after shaving | 90% |

1. A personal care article including a substrate having impregnated therein, applied thereon or arranged to substantially encase, a cosmetic composition which includes a hair modification active, wherein the pH of the composition is substantially less than 9.0, and at least a portion of the article has exfoliating properties.

2. An article according to claim 1, wherein the exfoliating properties result from the physical properties of the substrate.

3. An article according to claim 2, wherein the substrate provides exfoliating properties as a result of apertures therein, indentations therein or protrusions thereon.

4. An article according to claim 1, wherein the exfoliation properties result from at least one exfoliating component in the composition.

5. An article according to claim 4, wherein the exfoliating component includes one or more of a liquid wax, alpha hydroxy acids, beta hydroxy acids, or Silica agglomerates.

6. An article according to claim 4, wherein the exfoliating component breaks down on the skin.

7. An article according to claim 1, wherein the pH of the article is less than 8.5.

8. An article according to claim 1, wherein the pH is greater than 5.0.

9. An article according to claim 1, wherein the pH is in the range 7-8.

10. An article according to claim 1, wherein the one or more hair modification active is selected from one or more of a sulfur compound, potassium thioglycolate, sodium thioglycolate, ammonium thioglycolate, thioglycerol, thioglycol, thioxanthine, thiosalicylic acid, N-acetyl-L-cysteine, lipic acid, NaHSO₃, Li₂S, Na₂S, K₂S, MgS, CaS, SrS, BaS, (NH4)2S, sodium dihydrolipoate 6,8-dithiooctanoate, sodium 6,8-dithiooctanoate, salts of hydrogen sulfide, thioglycolic acid, thioglycerol, 2-mercaptopropionic acid, 3-mercaptopropionic acid, mercaptoethanol, dithioerythritol (DTE), thiomalic acid, calcium thioglycolate, guanidine thioglycolate, glyceryl monothioglycolate, monoethanolamine thioglycolate, monoethanolamine thioglycolic acid, diammonium dithiodiglycolate, ammonium thiolactate, monoethanolamine thiolactate, thioglycolamide, homo-cysteine, cysteine, acetyl cysteine, glutathione, dithiothreitol, dihydrolipoic acid, 1,3-dithiopropanol, thioglycolamide, glycerolmonothioglycolate, thioglycolhydrazine, keratinase, hydrazine sulfate, hydrazine disulfate triisocyanate, guanidine thioglycolate, calcium thioglycolate, cysteamine, Xyleine, lipoic acid, sodium dihydrolipoate, thiolactic acid, ammonium thiolactate, monoethanolamine thiolactate, thiopropionic acid, 2-thiolhistidene, 6-mercaptopurine, dimercaptosuccinic acid, thiophenol, 4-methoxythiophenol, 4-bromothiophenol, benzyl mercaptan, 2-mercaptobenzothiazole, bromohexine, carbocysteine, domiodol, erdosteine, letosteine, lysozyme, mecysteine hydrochloride, mesna, sobrerol, stepronin, tiopronin, tyloxapol, nor-dihydroguaiaretic acid (NDGA), wortmannin, resveratrol, thioglycolhydrazide, glutathione (reduced form), dimercaptosuccinic acid and/or Bowman Birk inhibitor (or one of its mutated forms).

11. An article according to claim 1, wherein each hair modification active is present in an amount less than 10% by weight of the composition.

12. An article according to claim 1, wherein the substrate is an absorbent or a porous material.

13. An article according to claim 1, wherein the substrate is non-woven woven material, thermo-bonded or air laid.

14. An article according to claim 1, which is flushable and/or biodegradable.

15. An article according to claim 1, which includes two or more layers.

16. An article according to claim 1, wherein the composition includes a surfactant.

17. An article according to claim 1, wherein the composition includes at least one accelerator.

18. An article according to claim 1, wherein the composition includes a dye and/or a fragrance.

19. An article according to claim 1, wherein the composition includes at least one skin conditioning active.

20. An article according to claim 1, wherein the skin conditioning active includes one or more of naturally derived oils and esters thereof, silicones, benzoates, alkanes, alkenes, alkyl alkanoates, cetearyl esters, acid glycol esters, alkylene glycols and poly-esters and poly-ethers thereof, glycerins, glycols, glycerides, glyceryl triesters, alcohols, alkoxylated alcohols, lactates, polysorbates, palmitates, succinates, laurates, oleates, tocopherol and esters thereof, alkyl acetates, linoelates, linoenate, myristates, sugars, citrates, carbonates, phosphates, dehydro-acetic acid, and/or stearates.

21. (canceled)

22. A method of modifying mammalian hair which method includes:
a) providing an article according to claim 1; and
b) permitting the article to contact the skin where hair modification is desired.

23. (canceled)

24. A method of reducing ingrowing hairs on or in mammalian skin which method includes:
a) providing an article according to claim 1; and
b) permitting the article to contact the skin to be treated.
